(19) United States
(12) Patent Application Publication
(10) Pub. No.: US 2006/0270984 A1
(43) Pub. Date: Nov. 30, 2006

(54) Automatic injection device

(76) Inventor: Edgar Hommann, Grossaffoltern (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/418,731

(22) Filed: May 4, 2006

Related U.S. Application Data
(63) Continuation of application No. PCT/CH04/00652, filed on Oct. 29, 2004.

(30) Foreign Application Priority Data
Nov. 5, 2003 (DE) 103 51 599.2

(51) Int. Cl. A61M 5/20

(52) U.S. Cl. 604/134

(57) ABSTRACT

An injection device including a needle guard element retracted in an initial state and extendable to protect a needle carried by the injection device.

### CROSS-REFERENCE TO RELATED APPLICATION(S)

This application is a continuation of International Application No. PCT/CH2004/000652, filed on Oct. 29, 2004, which claims priority to German Application No. 103 51 599.2, filed on Nov. 5, 2003, the entire contents of both of which are incorporated herein by reference.

### BACKGROUND

The present invention relates to devices for administering, delivering, injecting or dispensing substances, and to methods of making and using such devices. More particularly, it relates to such devices, including, for example, injection devices, injection pens, etc., for dispensing medicinal substances, such as insulin or hormones, in selected amounts or doses. More particularly, the present invention relates to an auto-injection device, which may be designed to be disposable and/or for a single use only.

FIG. 3 illustrates an injection device 10 with a needle 5 disposed in the injection device 10 and surrounded by a needle guard 1 and a needle guard cap 6. The needle guard 1 is biased in the forward or dispensing direction of the injection device 10, on the left-hand side of FIG. 3, by a needle guard spring 3 supported against the housing 14 of the injection device 10. To administer an injection, the needle guard cap 6 is removed. The injection device 10 is placed with the front end of the needle guard element 1 on the injection point and the needle guard element 1 must be pushed back against the force of the needle guard spring 3 by the indicated distance D in order to be able to insert the needle 5. The fact that the needle guard element 1 has to be pushed back against the force exerted by the needle guard spring 3 over the distance D, which is usually in the range of approximately 18 mm, means that a user has to push the injection device 10 onto the injection point, applying a relatively strong pressure, for quite a long time in order to administer the injection. If the ampoule 11 contains a substance or medicine which should not come in contact with metal—for example, the needle 5—for any length of time, it is necessary for the needle 5 to not be fitted on the ampoule 11 until shortly before the injection.

### SUMMARY

In one embodiment, the present invention comprises an injection device and a mechanism for priming the injection device, whereby the injection device may be placed in a ready-to-use state in which an injection can be safely administered while applying a light force.

In one embodiment, the present invention relates to a priming device or a tool for priming an injection device, for example an automatic injection device, which may be designed in the form of a disposable part for one-off use, in order to administer an injection. Typically, the injection device contains an ampoule containing a substance to be dispensed, such as a medicament, and the ampoule is closed, being provided with a protective cap or seal of a known type. Such known protective caps may consist of a rubber part sealing the ampoule, and the rubber part may be secured on the ampoule by a plastic cap which may also have a thread for a Luer needle, for example. In some preferred embodiments, the plastic cap is provided with a breaking point or area or line of weakness which can be broken by applying pressure, e.g., by turning, pulling, etc., so that the rubber part can be removed together with the broken-off plastic part to open the ampoule. If a connecting element for a needle is provided on the cap, a Luer needle can be screwed or fitted thereon. For the purpose of the present invention, the priming device or tool is already connected to or connectable to the seal or protective cap in the initial state, so that the protective cap of the ampoule can be removed by turning and/or pulling the priming device or the tool, thereby enabling a needle to be attached. This being the case, a priming device in accordance with one embodiment of the present invention can be inserted in the injection device or is already present in the injection device and, in the inserted state, is easy for a user to grasp so that it can be turned relative to the injection device to open the seal of the ampoule.

In one embodiment, the priming device can advantageously be pushed into or inserted in the dispensing or front end of the injection device and projects out of the injection device, as a result of which a user can easily take hold of the priming device and turn it relative to the injection device so that a cap of the ampoule opens and can be taken out of the injection device together with the priming device. In some embodiments, the priming device may be in the form of an approximately cylindrical, elongated element slightly smaller than the internal diameter of a needle guard element or the front end of the injection device.

In one embodiment, the present invention comprises a priming device or a tool for priming an injection device comprising a holder element for a needle, for example a Luer needle, which is able to hold the needle for fitting it on or inserting it in the injection device and releasing the needle after it is attached so that the priming device can then be taken out of the injection device. The injection device is thus made ready for administering an injection when a needle is fitted on an opened ampoule. In one embodiment, therefore, the priming device in accordance with the present invention enables a needle to be fitted or screwed on the injection device in a region thereof that is protected by a housing or a needle guard element and is difficult to access. In some embodiments, the needle is connected to the injection device or to an ampoule, for example by a screw fitting, and the fixture or connection of the needle to the priming device can be released by pulling the priming device off the needle connected to the ampoule or injection device. The holder of the needle may be a simple receiving element into which the needle is pushed, or may alternatively comprise releasable retaining or connecting elements, such as catch lugs, clamping pieces or clips, or the like. The priming device may be designed so that a needle is already fitted in the priming device in the initial state or, alternatively, it may be designed so that a needle is inserted into the priming device and can then be fitted on the injection device with the priming device.

In one embodiment, the priming device is designed so the needle incorporating a needle guard cap fitted on it can be inserted in or fitted on the priming device, in which case the priming device is able to hold the needle guard cap by means of a latching action with the needle guard cap. Thus, after the needle has been attached to the priming device, the needle can be attached to the injection device by using the priming device and the priming device can be removed together with the needle guard cap thereby exposing the needle in readiness for an injection.

In one preferred embodiment, the priming device comprises both the above-mentioned connecting element for connecting the priming device to an ampoule seal or to an ampoule protective cap, and the holder element for a needle described above, in which case these two elements are provided at opposite ends of generally rod-shaped priming device, for example, so that the priming device can easily be turned by 180° after the ampoule has been opened to fit a needle on the opened ampoule.

In some preferred embodiments, the priming device comprises a mechanism for limiting a torque which is transmitted to the injection device by a user when turning the priming device to screw on a needle. The torque can be limited by using a two-part design for the priming device, for example, in which one part of the priming device gripped by a user is coupled with the other part by means of connecting elements, such as cams or the like, so that when a specific and pre-definable torque is exceeded, the connection between the two parts of the priming device is released or broken, thereby enabling the two parts to rotate relative to one another. Thus, a torque acting on a first part can be transmitted to the second part only to a specific maximum degree and a higher torque causes the first part move, turn or slide relative to the second part, thereby ensuring that a needle can only be screwed onto the injection device with a specific maximum torque to prevent too high force or too high a torque from effecting the injection device.

In one embodiment, the priming device locks the injection device when inserted therein, i.e., when inserted it prevents an injection procedure from being initiated. In some embodiments, an injection can not be made until the injection device has been fitted with an injection needle in which case a locking ring (described in more detail below) may be provided, wherein the ring can not be removed from the injection device other than with the priming device.

In one embodiment, the present invention comprises a locking ring for an injection device, disposed on a dispensing end of the injection device and retained by a latch mechanism. The locking ring prevents a movement of a trigger element or sleeve for a triggering an injection procedure, such as a sliding movement. To this end, the locking ring is fitted to the injection device at the front end of the injection device, for example to the trigger sleeve and/or the needle guard, and is connected to the injection device by a releasable connection, such as one or more catch elements. In some embodiments, the connection between the locking ring and injection device is designed so that the latter can be released, in some instances by a priming device after a needle has been attached to the injection device. In some embodiments, the connection between the locking ring and injection device is not releaseable until an injection needle has been safely connected to the injection device, for example by inserting the priming device sufficiently deep in the injection device. To this end, cams or drivers may be provided on the external face of the priming device, to release or unlock the lock or connection between the locking ring and injection device after the priming device has been inserted sufficiently deeply, enabling the locking ring to be removed from the injection device.

In some embodiments, the locking ring is connected to the injection device so it is able to rotate relative ot the injection device, but can not slide axially until the connection between the locking ring and injection device has been unlocked or released by the priming device. Accordingly, the locking ring can be coupled with the injection device by means of grooves, whereby a rotation of the locking ring can be transmitted to the priming device, inserted through the locking ring into the injection device, thereby enabling a user to turn the priming device with the locking ring to open the cap or seal of an ampoule, or to attach or screw on an injection needle.

In some embodiments, the locking ring is mounted on the injection device so it is not able to move in the axial direction without a connecting or bearing element being released, and no axial displacement is possible to remove the locking ring until corresponding or complementary connecting or retaining elements have been released by a priming device as described above.

In one embodiment, the present invention comprises an injection device carrying a locking ring of the type described above, wherein the injection device comprises a trigger that can not be operated to trigger an injection as long as the locking ring is on the injection device.

Another embodiment of the present invention, which may be used in conjunction with but also independently of the elements and constituent components of an injection device or a priming device described above, comprises an injection device on which a needle can be screwed or fitted, in which a needle can be inserted, or which is already connected to a needle, and a needle guard element. The needle guard element is disposed in a retracted position in an initial state and can be extracted, extended or pushed out by a spring element after an injection procedure to cover or surround the needle thereby preventing inadvertent contact with the needle. In some embodiments, the needle guard can be moved from an initial position to an extracted or pushed-out position, and can then be secured or locked by a catch connection or pawl so that it can no longer be moved from the extracted position.

In some embodiments, the needle guard, which is not extracted or extended in the initial state, is movable as part of an injection sequence or procedure, whereby the injection device may be shorter than known injection devices with retractable needle guards that are extended or extracted in the initial state. The needle guard of the present invention, which is not extracted in the initial state, enables the release path for administering an injection to be made shorter, requiring less force, because the guard does not first have to be pushed back across a longer distance against a force caused by a needle guard spring to release the needle. In accordance with one embodiment of the present invention, the needle is first extracted or extended from the injection device, after which the needle guard is pushed over the extracted or extended needle.

In some embodiments, the needle guard is mounted on a syringe holder inside the injection device and is axially limited to a forward position by a stop. The needle guard is biased into the forward position by a spring element supported on the syringe holder.

In some embodiments, a trigger sleeve is disposed coaxially with the needle guard. The trigger sleeve projects beyond the needle guard and carries a locking ring of the type described above which prevents the trigger sleeve from inadvertently moving. The injection device can not be triggered until the trigger sleeve is displaced relative to the needle guard, which can be achieved by applying the injection device to an injection site. After it has been displaced or pushed in to the front edge of the needle guard, the trigger sleeve establishes a mechanical connection between a trigger button, previously inoperable to trigger an injection, and a trigger mechanism.

After the trigger button has been operated, an automatic triggering procedure is effected, whereby an injection needle is pushed forward or ejected from the injection device by the force of a spring element to enable it to pierce a tissue. A displacement body, for example a plug, piston or the like, is then pushed into an ampoule so that a substance contained in the ampoule is dispensed through the injection needle. After an injection procedure has been initiated and performed, and the injection device has been moved away from the injection site, the needle guard element is biased by a spring in the direction of the injection needle and is automatically pushed over the injection needle.

In some embodiments, the needle guard element may comprise an indicator, signal or display to indicate that the injection has been used. For example, a signal color, e.g., red, orange, yellow, etc., may be provided on the element In some embodiments, a locking element such as a catch may be provided to latch or lock the needle guard in the extracted position to protect the injection needle and to minimize the chance of accidental contact with the needle.

It should be appreciated that any of the embodiments, features, functions and/or structures described herein may be used cooperatively and/or to complement each other.

### BRIEF DESCRIPTION OF THE DRAWINGS

FIGS. 1A to 1F illustrate one embodiment of the present invention, an injection device which is primed by means of a priming tool, the device being in readiness to make an injection;

FIGS. 2A to 2F illustrate how an injection is made with an injection device in accordance with embodiments of the present invention, after which the injection needle is locked; and

FIG. 3 is a cross-section through an injection device.

### DETAILED DESCRIPTION

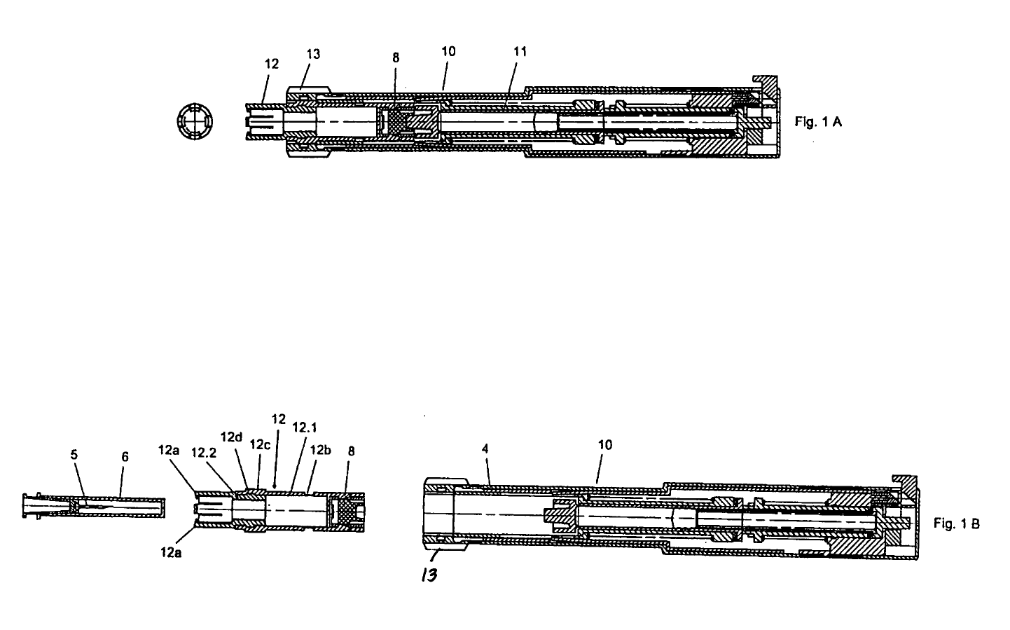

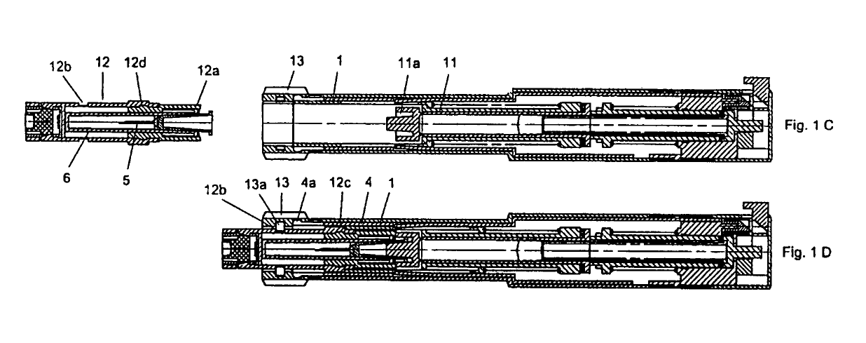

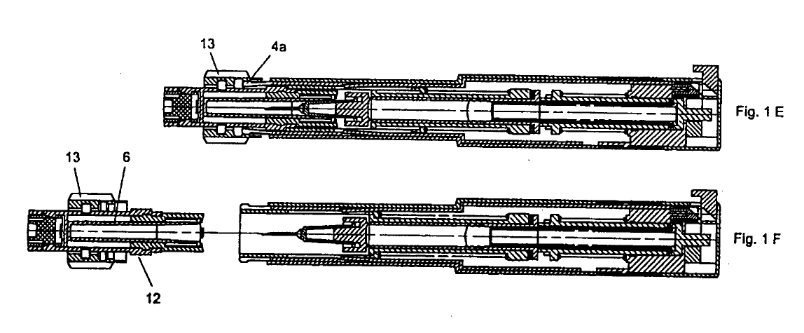

FIG. 1A depicts an automatic injection device 10, with an ampoule 11, closed by a protective cap 8 made from rubber. The cap 8 has a plastic potion which is connected to the ampoule 11 and also contains a thread for a Luer needle. The protective cap 8 is fixedly connected to the tool or priming device 12, which is mounted so as to be axially slidable and coupled in the locking ring 13 so as to rotate with it. By means of grooves or other suitable elements, the tool 12 is connected to the locking ring 13, which is mounted on the injection device 10 so as to rotate or is axially connected to it, so that a rotation of the locking ring 13 is transmitted to the tool 12, as a result of which a breaking point of the protective cap 8 is broken open, thereby opening the front end of the ampoule 11.

The tool 12 connected to the protective cap 8 can then be pulled out of the injection device 10, as illustrated in FIG. 1B. The locking ring 13 remains axially connected to the injection device 10.

At the end of the tool 12 lying opposite the end incorporating the protective cap 8, a Luer needle 5 with a needle guard cap 6 fitted on it can be inserted in the tool 12, and catch lugs 12a disposed on the insertion orifice of the tool 12 ensure that the needle guard cap 6 can not be taken out of the tool 12 again, as illustrated in FIG. 1C.

If the tool 12 is rotated by 180°, as illustrated in FIGS. 1C and 1D, the Luer needle 5 can be inserted into the injection device 10 through the locking ring 13 and screwed into an internal thread 11a provided on the ampoule 11 in order to connect the Luer needle 5 fixedly to the front end of the ampoule opening. As this happens, the locking ring 13 is coupled with the tool 12, for example by means of grooves, so that a rotation of the locking ring 13 can be transmitted to the tool 12 in order to screw on the Luer needle 5.

The tool 12 consists of 2 parts 12.1 and 12.2, which are connected so that only a specific maximum torque can be transmitted from one part to the other. The connection between the two parts 12.1 and 12.2, provided in the form of catch lugs for example, is released when a pre-defined maximum torque is exceeded and thus causes one part to rotate relative to the other, thereby ensuring that the needle 5 is screwed with only a defined torque, below a pre-definable maximum torque, for example by turning the locking ring 13 coupled with the tool 12, as illustrated in FIGS. 1D and 1E.

The tool 12 together with the locking ring 13 released from the injection device 10 by the tool 12 can then be removed from the injection device 10, so that the injection device 10 can be primed for carrying out an injection with the ampoule 11 open and the needle 5 fitted, as illustrated in FIG. F.

After the protective cap 8 has been removed, the needle 5 is placed on the ampoule 11, as illustrated in FIG. 1D. A cam or projection 12c disposed externally to the tool 12 pushes back the needle guard 1 disposed inside the trigger sleeve 4, thereby releasing a cam 4a on the external face of the trigger sleeve 4 which holds the locking ring 13 on the injection device 10. The cam 4a can be pushed radially inwards, thereby releasing the lock of the locking ring 13 so that the locking ring 13 can be removed from the trigger sleeve 4 and hence from the injection device 10, as illustrated in FIGS. 1E and 1F.

The locking ring 13 can be removed due to the fact that pre-tensioned spring elements 13a are provided in the locking ring 13, which push radially inwardly and latch in matching recesses or cut-outs 12b on the external face of the tool 12. As a result the locking ring 13 is fixedly connected to the tool 12 and is removed from the injection device 10 together with the tool 12 when the tool 12 is removed, as illustrated in FIG. 1F. The cut-outs 12b are advantageously disposed on the tool 12 so the tool 12 must be inserted in the injection device 10 at least so far that the needle 5 is reliably fitted or secured before the spring elements 13a of the locking ring 13 are able to latch.

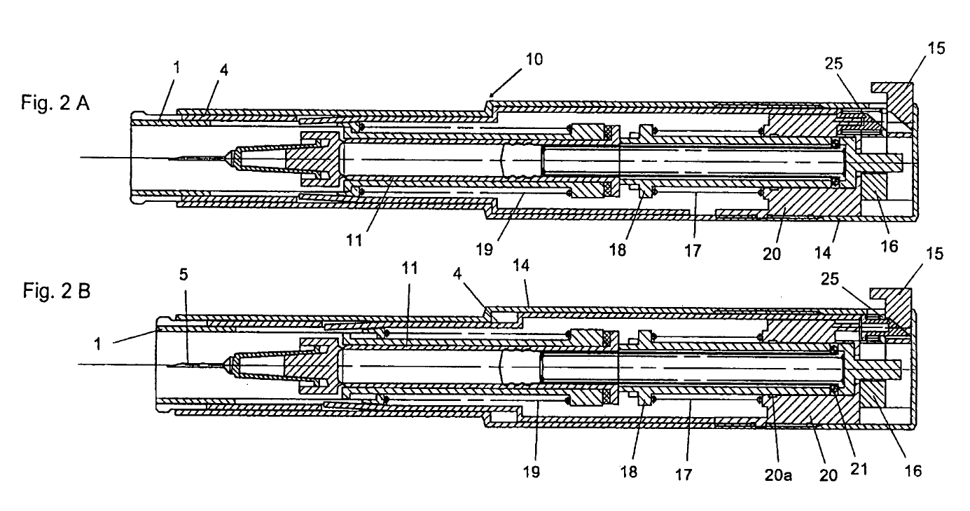

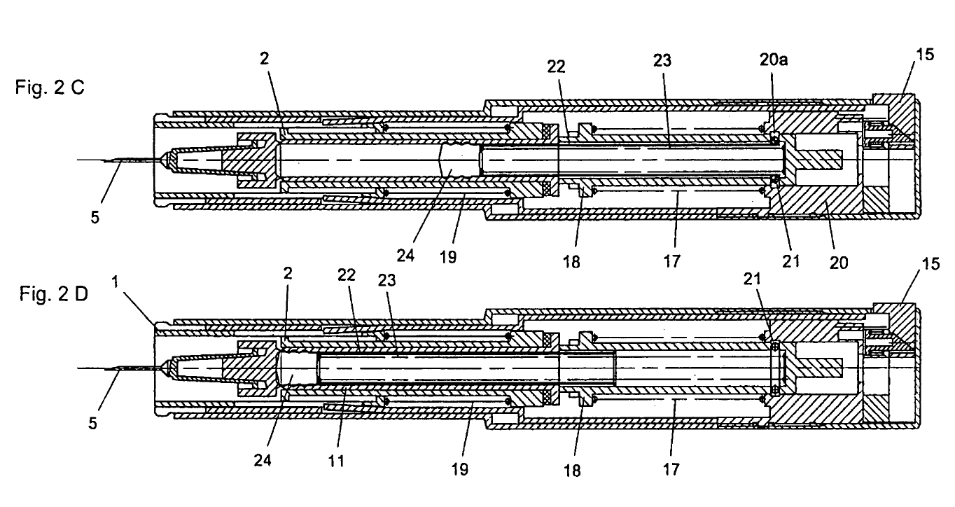

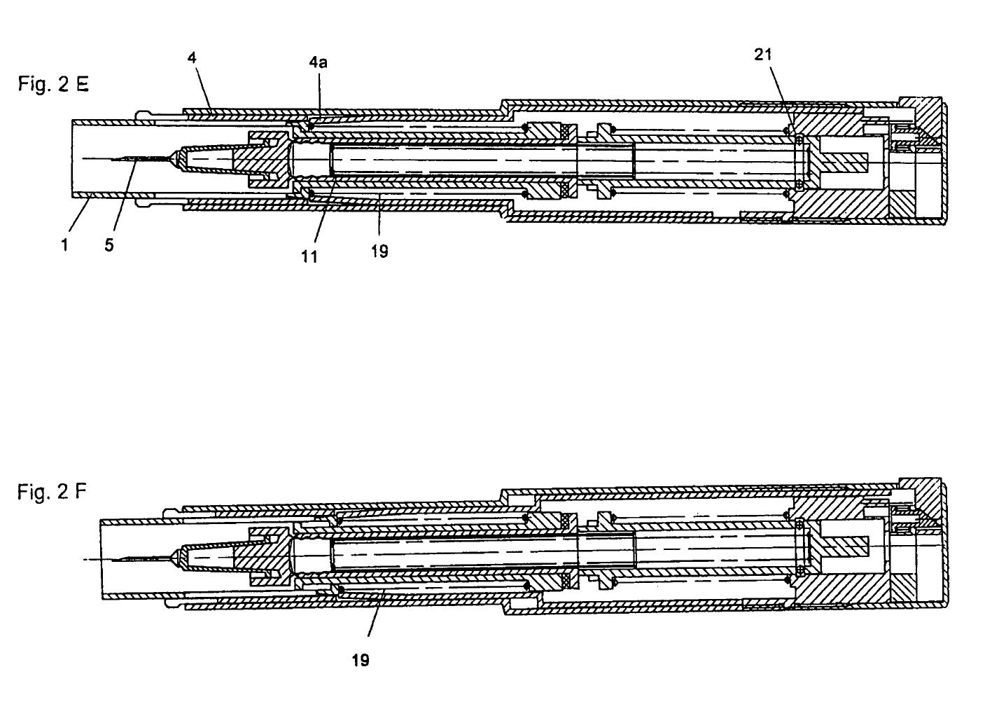

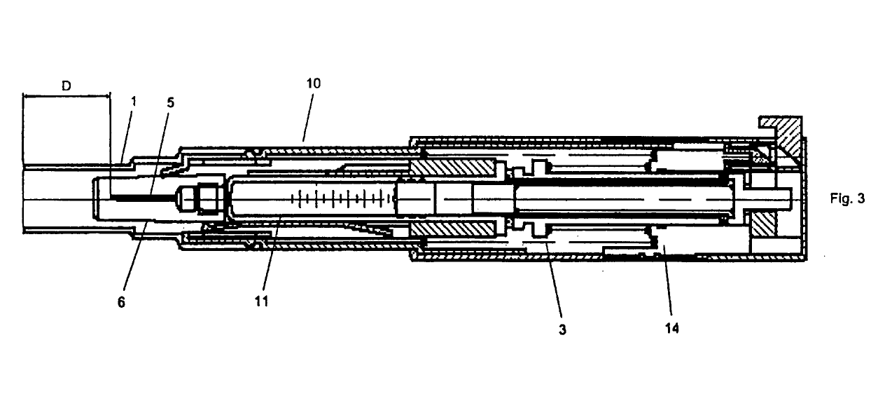

FIGS. 1F and 2A illustrate the injection device 10 primed in readiness for administering an injection.

When the injection device as illustrated in FIG. 2A is applied to an injection site with a light pressure, the trigger sleeve 4, together with the needle guard 1, is pushed back, as illustrated in FIG. 2B.

The procedure involved in administering or making an injection will be described with reference to FIGS. 2A to 2F. FIG. 2A illustrates the injection device 10 after the ampoule 11 has been opened and the needle 5 has been attached. The trigger sleeve 4 is in the initial state or has been positioned on an injection site so that it is pushed back until the front end of the trigger sleeve 4 lies more or less on the front end of the needle guard 1, as illustrated in FIG. 2A. When placed on an injection site, the trigger sleeve 4 is pushed into the ampoule housing 14 and, due to a displacement of the coupling element 25 illustrated in FIG. 2B, causes the trigger button 15, which until now has been in a position in which it could be depressed and released again without having any effect on the injection device 10, to be coupled with the needle locking element 16 so that when the trigger button 15 is depressed, the needle locking element 16 is pushed out of a position in which it locks the ampoule holder 18, and the ampoule holder 18 is pushed by the needle ejector spring 17, supported against the ampoule housing 14 or an ampoule holder guide 20, in the extraction direction of the injection needle 5, as a result of which the needle 5 together with the ampoule 11 disposed behind the needle 5 is extracted from the injection device 10, as illustrated in FIG. 2C. As a result, the syringe holder 2 is simultaneously pushed into a forward position, thereby tensing or compressing the needle guard spring 19 disposed between the syringe holder 2 and the needle guard 1.

When the ampoule holder 18 is pushed so far back in the ampoule holder guide 20 that the locking elements 21 lie opposite recesses or cut-outs 20a provided on the internal face of the ampoule holder guide 20, as illustrated in FIG. 2C, the locking elements 21, biased radially outwardly, are forced into the cut-outs 20a, causing the plug holder or slide 22 to be unlocked and released from the ampoule holder 18. See FIG. 2D. The injection spring 23 biased between the rear end of the ampoule holder 18 and the plug holder or slide 22 is therefore able to relax and pushes the plug 24 into the ampoule 11, as illustrated in FIG. 2D. As a result the substance contained in the ampoule 11 is displaced and dispensed through the extracted needle 5.

When the injection device 10 is moved away from the injection site after making the injection, the needle guard 1 is pushed forwards out of the injection device 10 and over the needle 5 by the needle guard spring 19 biased in the direction of the injection needle 5, as illustrated in FIG. 2E, thereby enabling the needle 5 to be blocked or covered. An inwardly biased catch or pawl 4a provided in the trigger sleeve 4 latches behind a rear stop of the needle guard 1, thereby locking it in the extracted position.

In one embodiment, as illustrated in FIG. 2F, the needle guard can be pushed by an extra distance by applying a pressure, but without the needle 5 being exposed, until the trigger sleeve 4 coupled with the needle guard 1 sits on a projection of the ampoule holder guide 20.

If the needle guard or the external face of the needle guard 1 has a signal color or colored area, the extracted needle guard 1 can simultaneously also be used as an indicator to indicate that the automatic injection device has already been used and can not be used again.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device including a needle guard element retracted in an initial state and extractable to protect a needle carried by the injection device.

2. The injection device as claimed in claim 1, further comprising a trigger sleeve disposed generally coaxially with the needle guard element, wherein the trigger sleeve is displaceable relative to the needle guard element to permit a triggering procedure.

3. The injection device as claimed in claim 2, wherein the trigger sleeve is either outside or inside the needle guard element.

4. The injection device as claimed in claim 2, further comprising a coupling element, wherein the trigger sleeve co-operates with the coupling element to operably couple a trigger button and a needle locking element to enable an injection procedure to be initiated.

5. The injection device as claimed in claim 4, wherein the needle guard element comprises a display.

6. The injection device as claimed in claim 4, further comprising a catch for latching the needle guard in an extracted position.
